(19) United States
(12) Patent Application Publication
(10) Pub. No.: US 2004/0220747 A1
(43) Pub. Date: Nov. 4, 2004

(54) Method and apparatus for positionally correcting data in a three dimensional array

(75) Inventor: Bert (Berton) Gunter, Princeton, NJ (US)

Correspondence Address: WOODCOCK WASHBURN LLP, ONE LIBERTY PLACE, 46TH FLOOR, 1650 MARKET STREET, PHILADELPHIA, PA, 19103, US

(73) Assignee: Merck & Co., Inc.

(21) Appl. No.: 10/861,187

(22) Filed: Jun. 4, 2004

Related U.S. Application Data
(62) Division of application No. 09/632,422, filed on Aug. 4, 2000.
(60) Provisional application No. 60/217,772, filed on Jul. 12, 2000.

(51) Int. Cl. G06F 19/00; G01N 33/48; G01N 33/50

(52) U.S. Cl. 702/19; 702/20

(57) ABSTRACT

An assay is performed such that a compendium of raw assay data is developed and is then positionally corrected. The assay comprises a plurality of longitudinally oriented plates p, each having a wells organized into rows i and columns j. Each well (i, j, p) has a raw value xᵢⱼₚ associated therewith that is deconstructed into: a plate effect value representing extraneous effects attributable to the plate p of the well (i, j, p); a row effect value representing extraneous effects attributable to the row i on the plate p of the well (i, j, p); a column effect value representing extraneous effects attributable to the column j on the plate p of the well (i, j, p); a non-additive, interaction effect representing extraneous positional effects attributable to consistent positional effects beyond the plate, row, and column effects previously determined for the (i, j, p) well on plate p; and a residual data value that is left over once all the above extraneous effects are taken into account. Thereafter, the residual data value associated with each well (i, j, p) is employed to represent the well (i, j, p) as compared with all other wells (i, j, p) on the plate p.

### CROSS-REFERENCE TO RELATED APPLICATION

This application is a divisional of U.S. patent application Ser. No. 09/632,422, filed Aug. 4, 2000 which claims the benefit of U.S. Patent Application No. 60/217,772, filed Jul. 12, 2000 and entitled “METHOD AND APPARATUS FOR POSITIONALLY CORRECTING DATA IN A THREE DIMENSIONAL ARRAY”, hereby incorporated by reference.

### FIELD OF THE INVENTION

The present invention relates generally to an apparatus for and method of positionally correcting data in a three dimensional array in data sets that are generated from output functions which are subject to variations due to positional effects.

### BACKGROUND OF THE INVENTION

Drug discovery is often achieved through in vitro assays employed to identify compounds that have effects on various specific biological processes. Efforts have been undertaken to identify agents that may block, reduce, or even enhance the interactions between biological molecules.

It is well known that the interaction between a receptor and its ligand often may result, either directly or through some downstream event, in either a deleterious or beneficial effect on a biological system which can thus affect a patient with a condition, disease or disorder associated with activity in such biological system. Accordingly, agents which can reduce, block or enhance interaction between a receptor and its ligand are sought as pharmacologically active entities.

Similarly, it is well known that the enhancement or inhibition of enzyme activities often may result, either directly or through some downstream event, in either a deleterious or beneficial effect on a clinically relevant biological system. Accordingly, efforts are undertaken to identify compounds that serve as substrates, inhibitors or catalysts for enzymatic reactions using in vitro assays.

In addition to these examples, there are many other drug targets and biological systems for which in vitro assays can and are used to identify pharmacologically active and biologically active agents. For example, human genome research has also uncovered large numbers of new target molecules against which the efficacy of test compounds may be screened.

One strategy employed in modern drug discovery is to maximize the throughput of the assays that are used to screen test agents which possess a desired pharmacological activity. In particular, by screening a large number of different test agents, the probability of testing and identifying a compound with the desired activity is greater. Using robotics and other automation technology together with automated detection systems, high throughput screening assays have been developed which apply industrial/manufacturing concepts and design to research protocols.

High throughput screening assays provide for the performance of multiple identical assays in parallel on a platform matrix. Multiple parallel assays are performed sequentially. By sequentially performing multiple parallel assays, the data outputted may be compiled and arranged in a three dimensional array.

Sequential and parallel processing in high throughput screening methods using robotics and automation allows for the ability to test many thousands of test agents in an assay. Automated screening procedures allow high throughput evaluation of individual test agents in collections or libraries which contain large numbers of test agents in order to assess functional biological/pharmacological properties of each test agent.

Screening of collections of test agents is an important aspect of efforts to identify lead compounds that have pharmacological activity and that can be further developed into new drugs and therapeutic compositions. Such test agents include but are not limited to chemically synthesized molecules, including libraries of compounds synthesized by combinatorial chemistry; natural products, including cells, cell extracts, nucleic acid molecules, cell culture media, proteins, isolated genetic material, fungal extracts and microbial fermentation broths; and recombinant products such as viral and phage particles, proteins and peptide libraries. Generally, the type of test agent used in the high throughput screening includes any composition or molecule which can be used in an in vitro assay. Most commonly, high throughput screening is performed using collections, also referred to as libraries, of test agents that include thousands of individual chemical entities or compositions.

High throughput screening procedures provide the ability to perform large numbers of identical functional assays that are predictive of bioactivity in a fully integrated automated format that accelerates data collection and lead identification, while also cutting costs. Each hit, i.e. test agent that produces a positive assay result, represents a lead candidate compound that has pharmacological activity. Lead candidate compounds may then be further investigated for development.

The assays used in high throughput screening procedures include any detectable activity with pharmacological or biological significance. Activities which are assessed in high throughput screening procedures include, but are not limited to, enzyme activation, enzyme inhibition, ligand-receptor binding, ligand-receptor binding inhibition, cell cycle inhibition, cell cycle activation, cell growth, cell division, cell activation, cell inhibition, activation of production of and/or release of cellular factors, inhibition of production of and/or release of cellular factors, ion pump, transport or channel activity, ion pump, transport or channel inhibition, activation of DNA synthesis, inhibition of DNA synthesis, activation of RNA synthesis, inhibition of RNA synthesis, activation of protein synthesis, inhibition of protein synthesis, metabolic activity, metabolic inhibition, activation of apoptosis, and inhibition of apoptosis.

High throughput screening procedures can be used to identify agents useful to treat infectious diseases such as compounds with anti-viral activity such as those that inhibit viral attachment to cells, infection, viral gene expression, viral gene replication, and viral particle assembly; antibiotic activity including anti-bacterial activity, anti-fungal activity, and anti-parasitic pathogen activity. High throughput screening assays are used in the search to identify pharmacologically active agents for use in medical and nutritional treatments and regimens such as, but not limited to, anti-cancer agents, anti-inflammatory agents, immunosuppressive agents, neuropharmacologically active agents, blood chemistry modifying agents, and agents for treatment of cardiac, pulmonary, renal, hepatic, pancreatic, bone, blood, gastrointestinal, and dermatalogical diseases.

Those skilled in the art routinely apply high throughput screening technology to identify active agents in a variety of different chemical and biological systems using a variety of target and reactions. Although drug discovery is a common application of high throughput screening, the present invention is useful in the field of high throughput screening data analysis generally.

In such assays, activation and inhibition can be detected and measured by detection and/or measurement of various detectable markers, such as but not limited to, those which are detected by their radioactivity, characteristics which can be observed optically or by electromagnetic detection, scintillation counting, fluorescence, visible dye changes in intracellular concentration of ionized calcium, cAMP or pH, trans-membrane potential and other physiological and biochemical characteristics of living cells which can be measured by a variety of conventional means, for example using specific fluorescent, luminescent or color developing dyes and the like.

High throughput screening is often performed using collections of test agents that are individually dispensed in wells of multi-well plates. Standard plates usually contain 96 wells (organized into an 8×12 array) while some larger plate sizes contain 384 wells (16×24 array), 1536 wells (32×48 array) and 3456 wells (48×72 array). Although these configurations are among the most common employed, other arrangements are equally useful in the present invention. Likewise, microchip array technology provides for the deposition of libraries of combinatorial chemical and biological materials in fixed two-dimensional arrays. Importantly, whether the platform is a microtitre plate, a microchip array or some other platform for performing parallel assays on a collection of individual test agents, the assay sites which contain test agents are arranged in identical matrices.

To assess the activity or inhibitory effect that a test agent has in an assay, positive and negative control samples are provided. Such controls are test samples that include the presence or absence of an active compound. These positive and negative controls provide data to which the results of test assays can be compared in order to determine the activity of the test agents.

As may be appreciated, several problems are associated with the analysis of data generated in high throughput screening assays. Such problems arise from variability in controls, variability in samples, and systemic variability, among other things.

A significant problem experienced in high throughput screening is that of positional effects which exist with respect to the location of wells on each plates. That is, a variability of background exists that may be associated with specific well locations within the matrix of a plate, where such variability is substantially consistent across a series of plates.

The variability of background due to positional effects has been observed to be sufficient to be responsible for a significant number of false positives and false negatives relative to controls. That is, data from test assays at specific well locations is consistently identified as being higher or lower for the parameter measured relative to positive and negative control data when compared to corresponding data from other well locations. The phenomenon of positional effects based upon well location on plates represents a real and significant problem to the predictability of data from high throughput screening.

False negatives result in the failure to identify a lead candidate compound which has the desired pharmacological activity from the library of chemical compounds being tested. Such failure to identify is of course a missed opportunity to further examine the compound and perhaps identify pharmacological relevance for the compound.

False positives result in further investigation and development of a compound which does not actually have the desired pharmacological activity. The further testing of false positives is an ineffective use of manpower and other resources as well as a waste of valuable stock from a chemical collection.

Accordingly, there is a need for an improved method and apparatus for analyzing high throughput screening data, a need for an improved method and apparatus for reducing the number of false positives and false negatives in high throughput screening assays, a need for an improved method and apparatus for correcting high throughput screening data for positional effects, and a need for an improved method and apparatus for analyzing assay conditions in high throughput screening using data analysis.

### SUMMARY OF THE INVENTION

The present invention satisfies the aforementioned need by providing a method of obtaining and evaluating assay data. In the method, an assay is performed such that a compendium of raw assay data is developed. The raw assay data is compensated for systematic and positional effects, the compensated data is scored, and the scored data is formatted according to a determined format.

In addition, the present invention provides a method of positionally correcting raw assay data from an assay comprising a plurality of longitudinally oriented plates p. Each plate p has a plurality of wells organized into rows i and columns j. Each well (i, j, p) has a raw value xᵢⱼₚ associated therewith, where the raw values xᵢⱼₚ comprise the raw assay data. Each raw value xᵢⱼₚ of an associated well (i, j, p) is deconstructed into:

a plate effect value representing extraneous effects attributable to the plate p of the well (i, j, p);

a row effect value representing extraneous effects attributable to the row i on the plate p of the well (i, j, p);

a column effect value representing extraneous effects attributable to the column j on the plate p of the well (i, j, p);

a non-additive, interaction effect representing an extraneous positional effect beyond the plate, row, and column effects previously determined for the (i, j, p) well on plate p; and

a residual data value that is left over once all the above extraneous effects are taken into account.

Thereafter, the residual data value associated with each well (i, j, p) is employed to represent the well (i, j, p) as compared with all other wells (i, j, p) on the plate p.

### BRIEF DESCRIPTION OF THE DRAWINGS

The foregoing summary as well as the following detailed description of the present invention will be better understood when read in conjunction with the appended drawings. For the purpose of illustrating the invention, there are shown in the drawings embodiments which are presently preferred. As should be understood, however, the invention is not limited to the precise arrangements and instrumentalities shown. In the drawings:

FIG. 1 is a flow chart detailing steps performed in formulating assay results in accordance with one embodiment of the present invention;

FIG. 2 is a block diagram showing a computer on which the steps detailed in FIG. 3 may be performed; and

FIG. 3 is a flow chart detailing steps performed in positionally correcting assay data in accordance with one embodiment of the present invention.

### DETAILED DESCRIPTION OF PREFERRED EMBODIMENTS

Certain terminology may be used in the following description for convenience only and is not considered to be limiting. For example, the words “left”, “right”, “upper”, and “lower” designate directions in the drawings to which reference is made. Likewise, the words “inwardly” and “outwardly” are directions toward and away from, respectively, the geometric center of the referenced object. The terminology includes the words above specifically mentioned, derivatives thereof, and words of similar import.

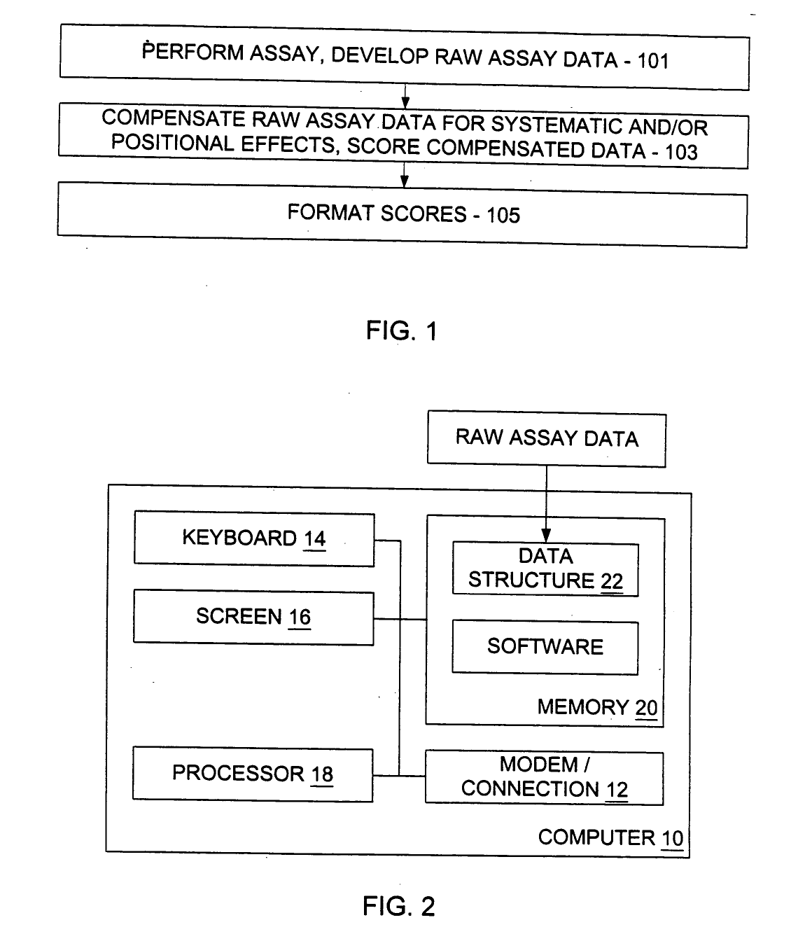

Generally, in the present invention, assay screening data is positionally corrected and then formatted into a form where such positionally corrected data may be presented to an appropriate assay analyst or the like. Thus, and referring to FIG. 1, now, the assay is performed such that a compendium of raw assay data is developed (step 101). Such raw assay data may be in any appropriate form without departing from the spirit and scope of the present invention. For example, such raw assay data may be expressed in terms of its original units of measure, or may be scaled based on an appropriate numerical scale (0-1, 0-100, −100-+100, etc.).

Preferably, the raw data is in a computer-readable form for purposes of allowing a computer-based algorithm to operate on such data, although such raw data may also be transcribed or otherwise converted into a computer-readable form without departing from the spirit and scope of the present invention. Any appropriate computer-readable form may be employed without departing from the spirit and scope of the present invention. For example, the computer-readable form may be an ASCII delimited file, a spreadsheet file, a table file, a database file, or the like. Presumably, the computer-readable form of the raw assay data is accessible by any particular software employed to perform the algorithm, as described below.

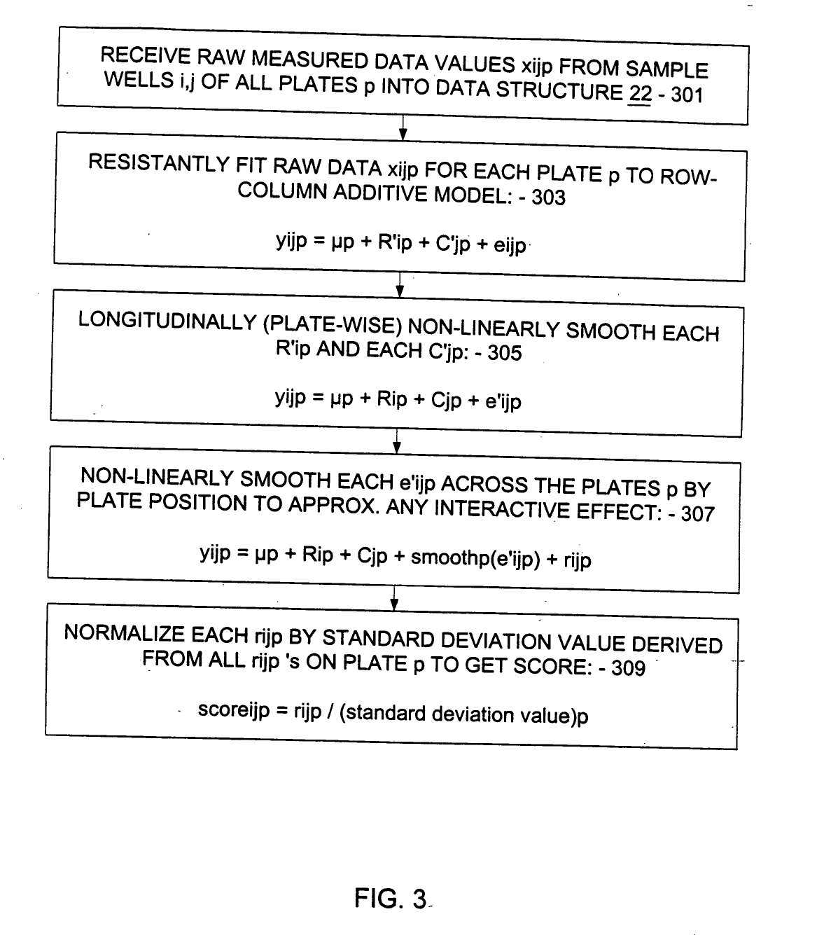

Once the raw assay data is developed and is in the computer-readable form, an appropriate algorithm is employed to process the raw assay data and compensate such raw assay data for systematic and/or positional effects (step 103). Such algorithm is described in more detail below in connection with FIG. 3. In the processing and compensating, the algorithm estimates background effects such as those that derive from a well being on a particular plate, being in a particular row on a plate, being in a particular column on a plate, being in a particular part of a series of plates, etc. In addition, the algorithm adjusts the compensated raw data for variations from plate to plate to result in a score value for each well. Note that although the algorithm as disclosed herein orients wells in terms of rows and columns on a plate, any other appropriate orientation system may be employed without departing from the spirit and scope of the present invention.

Finally, now that all systematic/positional/background effects have been removed from the raw assay data and such raw assay data has been scored to result in a score value for each well, such score values for all the wells may then be compared/organized/ranked and otherwise formatted into an appropriate form (step 105). Any appropriate formatting form may be employed without departing from the spirit and scope of the present invention. For example, each well may be ranked in a list according to its potency as represented by the score value for such well. Such formatted score values are then available for presentation to an appropriate assay analyst or the like.

Positionally Correcting Algorithm:

In one embodiment of the present invention, for purposes of performing the algorithm by which positional correcting takes place, the assay is performed in connection with a series of plates, where each plate is serially assayed. Thus, each plate has a time aspect or is ‘longitudinally’ positioned with respect to the other plates. Accordingly, each plate p in the series of plates is indexed by its order within the series:

p=1, 2, . . , P.

Likewise, for each plate p, each row i thereon and each column j thereon is indexed by its respective order on the plate:

i=1, 2, . . . , I; and

j=1, 2, . . . , J.

Thus, the raw measured data value as obtained from the assay for any particular well at row i and column j on plate p is xᵢⱼₚ. Of course, any other appropriate positional identification system may be employed without departing from the spirit and scope of the present invention.

In one embodiment of the present invention, the raw measured data values from the assay are employed to fit each raw measured data value to a model. In the model, each raw measured data value xᵢⱼₚ from a well (i, j, p) is deconstructed into:

a plate effect value representing extraneous effects attributable to the plate p of the well (i, j, p);

a row effect value representing extraneous effects attributable to the row I on the plate p of the well (i, j, p);

a column effect value representing extraneous effects attributable to the column j on the plate p of the well (i, j, p);

a non-additive, interaction effect representing extraneous positional effects attributable to consistent positional effects beyond the plate, row, and column effects previously determined for the (i, j, p) well on plate p; and

a residual data value that is left over once all the above extraneous effects are taken into account.

As should be appreciated, the residual data value more truly represents the potency of the sample in the well (i, j, p) as compared with all other wells (i, j, p) on the plate p. Accordingly, the purpose of the model is to obtain such residual data value.

Re-stated in more mathematical terms, the model fit by the algorithm of the present invention is:

xᵢⱼₚ=μₚ+Rᵢₚ+Cⱼₚ+smoothₚ(eᵢⱼₚ)+εᵢⱼₚ

where xᵢⱼₚ is the aforementioned raw measured data value for the well at row i and column j on plate p, and where εᵢⱼₚ is the possible systematic interaction effect for the (i, j) well on plate p. That is, εᵢⱼₚ is ‘the residual data’ left over after all possible positional effects (discussed immediately below) have been removed from the raw measured data value. Note that all the non-potent εᵢⱼₚ values for a plate p—expected to be the bulk of the data—are expected to vary about 0 in a generally Gaussian manner, while the potent εᵢⱼₚ values will differ greatly from 0.

The element μₚ in the above equation represents the overall median of all the raw measured data values of the wells on plate p (i.e. ‘the plate median’). Thus, the plate median represents the possible systematic measurement plate offset for plate p. Similarly, Rᵢₚ is the median of all the raw values of the wells for row i on plate p (i.e., ‘the row effect’) after taking into consideration the plate median, and Cⱼₚ is the median of all the raw values of the wells for column j on plate p (i.e., ‘the column effect’) after taking into consideration the plate median. Thus, the row and column effects represent the possible systematic measurement row offset for row i on plate p and the possible systematic measurement column offset for column j on plate p.

The element smoothₚ(eᵢⱼₚ) is the possible systematic measurement longitudinal offset for the (i, j) well on plate p (i.e., ‘the longitudinal effect’ and ‘the non-additive interaction’). As will be discussed in more detail below, smoothₚ(eᵢⱼₚ) results from a smoothing function, and is employed to take into account longitudinal position effects by combining data over similar plates to determine this effect. That is, it is expected that systematic positional effects like edge effects or a ‘high’ region on a plate would be fairly consistent from plate to plate, especially for plates that are measured close together in time sequence. Therefore, the model takes advantage of the information in “nearby” plates to “average” results from wells in the same (i, j) position after correcting for the additive effects of plate, row and column so that the corrected data are expected to be effectively similar up to measurement error.

The underlying assumption incumbent in the above-specified model is that almost all the wells (i, j, p) can be assumed to contain zero or low potency compounds, with only a small proportion of wells (i, j, p) containing high potency compounds of interest. Accordingly, resistant statistical methods that ignore the high potency ‘outliers’ are used to fit the model. The fitted model thus should capture the systematic positional measurement effects incumbent in any assay, while the ‘residual data’—the εᵢⱼₚ's—should contain the leftover non-systematic noise, including the aforementioned high potency outliers.

Commercially available statistical software may be employed to implement many of the functions of the algorithm of the present invention. Such software may for example include S-PLUS statistical data analysis software, produced and/or marketed by MATHSOFT, Inc. of Cambridge, Mass., although any other appropriate software may be employed without departing from the spirit and scope of the present invention. Examples of S-PLUS code written for the S-PLUS software and employed to implement the positionally correcting algorithm of the present invention are set forth in the attached Appendix. As should be appreciated by the relevant public, such S-PLUS software and other similar software include analyzing procedures as discussed for example in Exploratory Data Analysis, Tukey, John W., Addison-Wesley: Reading, Mass. (1977), which is hereby incorporated by reference.

Referring now to FIG. 2, such software may be operated in the form of modules or otherwise on any appropriate computer 10 without departing from the spirit and scope of the present invention. As is typical, such computer 10 may include appropriate computer components including a data entry device such as a modem or network connection 12, a keyboard 14, a data viewing device such as a screen 16, a processor 18, and memory 20, among other things.

In any case, the algorithm of the present invention proceeds as follows. Preliminarily, the raw measured data values xijp from the sample wells of all the plates are inputted/received into a data structure 22 in the memory 20 of the computer 10 (step 301 of FIG. 3). Such data structure 22 may be any appropriate data structure without departing from the spirit and scope of the present invention.

Thereafter, for each plate p, the raw data xᵢⱼₚ for such plate p is resistantly fit to a row-column additive model (step 303):

yᵢⱼₚ=μₚ+R′ᵢₚ+C′ⱼₚ+eᵢⱼₚ.

where:

yᵢⱼₚ=the raw measured data value for the well at row i and column j on plate p, as obtained from the data structure of step 301;

μₚ=the overall “average” for plate p (i.e. ‘plate effect’), as computed;

R′ᵢₚ=the possible systematic measurement row offset for row i on plate p (i.e., ‘row effect’), as computed;

C′ⱼₚ=the possible systematic measurement column offset for column j on plate p (i.e., ‘column effect’), as computed; and

eᵢⱼₚ=the residual data without taking into account any longitudinal/interactive effect.

In one embodiment of the present invention, the Tukey two way resistant median polish procedure is employed for such resistant fit, although any other appropriate procedure may be employed without departing from the spirit and scope of the present invention. As is known, such Tukey median polishing procedure is coded into and available from the aforementioned S-PLUS software. Accordingly, since such procedure is known to those in the relevant public, further discussion and explanation thereof need not be provided herein. Suffice it to say that given the raw measured data values from the data structure, such procedure employs an iterative procedure to result in a standard resistant row/column additive fit, thereby solving for each μₚ, R′ᵢₚ, C′ⱼₚ, and eᵢⱼₚ.

An issue arises, though, in the situation where, for example, there happens to be three potent wells in a column. This is a rare occurrence, but can and does nevertheless happen. Suppose also that such column with three potent wells has eight wells total, two of which are empty. Thus, such column has six wells containing samples, three of which have outliers representing potent samples. In such a situation, the C′ⱼₚ column effect for such column would be affected by the outliers, which is not desired. As should now be appreciated, the C′ⱼₚ column effect should contain errant column-based positional data, not actual non-positional data representing potent compounds.

In one embodiment of the present invention, to assure that multiple outliers in a column or a row do not overly affect column and row effect calculations, each R′ᵢₚ and each C′ⱼₚ is longitudinally (plate-wise) non-linearly smoothed so that their values in any plate p cannot be much different from nearby plates (step 305). Assuming that the same hit and miss pattern of values does not repeat in nearby plates, which is essentially a certainty, such longitudinal C′ᵢₚ and R′ⱼₚ smoothing results in the transfer of potent well effects to the residual data, where they belong.

Non-linear smoothing is known to those in the relevant public, and accordingly further discussion and explanation thereof need not be provided herein. Suffice it to say that given, for example, a series of R′ⱼₚ's from adjacent plates P_{X}, P_{X+1}, P_{X+2}, P_{X+3}, P_{X+4}, P_{X+5}, etc.:

R′ⱼₚ, R′ⱼₚ₊₁, R′ⱼₚ₊₂, R′ⱼₚ₊₃, R′ⱼₚ₊₄, R′ⱼₚ₊₅, etc.,

the smoothed R′ⱼₚ (Rⱼₚ) may for example be the median of R′ⱼₚ₋₁, R′ⱼₚ, and R′ⱼₚ₊₁. As may be appreciated, other smoothing functions, both simple and complex, may be employed. In fact, any appropriate smoothing function may be employed without departing from the spirit and scope of the present invention.

The amount of longitudinal smoothing of row and column effects necessary depends on the unknown true situation, and is therefore difficult to determine with certainty. However, for present purposes, a minimal amount will suffice because the residuals are themselves longitudinally smoothed, as will be discussed below, and a fairly rough estimate of the row and column effects is all that is needed.

In one embodiment of the present invention, a Tukey-type running median smoother is employed for such longitudinal row effect and column effect smoothing, although any other appropriate smoother may be employed without departing from the spirit and scope of the present invention. In one embodiment of the present invention, the smoother is 4(3RSR)2 H with the “twicing” option set to False. This results in a somewhat “rough” smooth. As is known, such Tukey-type running median smoother is coded into and available from the aforementioned S-PLUS software.

The result of such longitudinal row effect and column effect smoothing is that the un-smoothed R′ᵢₚ and C′ⱼₚ values in the previous equation are substituted with smoothed Rᵢₚ and Cⱼₚ values as calculated by the smoother. Thus, the residual value eᵢⱼₚ in the same equation is adjusted by the smoothing to e′ᵢⱼₚ:

yᵢⱼₚ=μₚ+Rᵢₚ+Cᵢₚ+e′ᵢⱼₚ.

Once e′ᵢⱼₚ has been derived for each well (i, j) of each plate p, such e′ᵢⱼₚ's are then non-linearly smoothed across the plates p by plate position. That is, the smoothing process is performed longitudinally for each well (i, j) to approximate any interactive effect (step 307), resulting in:

yᵢⱼₚ=μₚ+Rᵢₚ+Cⱼₚ+smoothₚ(e′ᵢⱼₚ)+rᵢⱼₚ.

where smoothₚ(e′ᵢⱼₚ) is the possible systematic measurement longitudinal offset (non-additive interactive offset) for the (i, j) well on plate p (i.e., the interactive effect’), and rᵢⱼₚ is the residual data left over after taking into account any interactive effect, as calculated.

Such interactive effect is approximated by longitudinal smoothing because no replicates are available in that each sample is tested only once in one well. Such approximation is thus achieved by assuming that nearby plates (longitudinally) are “pseudo-replicates” after correcting for their plate, row and column effects and combining the results by longitudinal smoothing. Nevertheless, such longitudinal smoothing is conceptually calculating a background positional effect on the plate p beyond that attributable to the row/column additive effects. Although perhaps ‘a cheat’, the smoothing works reasonably well to detect and compensate for consistent (over many plates) background positional effects.

Once again, non-linear smoothing is known to those in the relevant public, and accordingly further discussion and explanation thereof need not be provided herein. Suffice it to say that given, for example, a series of e′ᵢⱼₚ's from adjacent plates P_{X}, P_{X+1}, P_{X+2}, P_{X+3}, P_{X+4}, P_{X+5}, etc.:

e′ᵢⱼₚ, e′ᵢⱼₚ₊₁, e′ᵢⱼₚ₊₂, e′ᵢⱼₚ₊₃, e′ᵢⱼₚ₊₄, e′ᵢⱼₚ₊₅, etc.,

the smoothed e′ᵢⱼₚ (rᵢⱼₚ) may for example be the median of e′ᵢⱼₚ₋₁, e′ᵢⱼₚ, and e′ᵢⱼₚ₊₁. As may be appreciated, other smoothing functions, both simple and complex, may be employed. In fact, any appropriate smoothing function may be employed without departing from the spirit and scope of the present invention.

As may be appreciated, then, the result of step 307 is that each e′ᵢⱼₚ is deconstructed into smoothₚ(e′ᵢⱼₚ) and rᵢⱼₚ. In one embodiment of the present invention, the aforementioned Tukey-type running median smoother is employed for such longitudinal smoothing, although any other appropriate smoother may be employed without departing from the spirit and scope of the present invention. In one embodiment of the present invention, the smoother is 4(3RSR)2 H. As may be appreciated, the advantage of such smoother is that it does not tend to hide outliers—potent wells. In contrast, other commonly used time series filtering techniques that are essentially weighted averaging procedures can and do hide such outliers.

Considering the last equation, now, it is seen that the terms μₚ, Rᵢₚ, Cⱼₚ, and smoothₚ(e′ᵢⱼₚ) represent the fit and contain the systematic positional effects (plate, row, column, and longitudinal/interactive), if any. Thus, rᵢⱼₚ is the residual and represents the true relative potency of the well (i, j, p) as compared to all other wells (i, j, p) on the plate p—including extreme potencies of active compounds—without the distortion of the positional effects.

However, to compare potencies across plates p, i.e. across the entire assay, it is necessary to normalize each rᵢⱼₚ. That is, it must be remembered that all the rᵢⱼₚ values for a plate p are expected to vary about 0 in a generally Gaussian manner. It must also be remembered, though, that as between plates p, the Gaussian spread can and does differ, and must be taken into account when comparing potencies across such plates. Accordingly, in one embodiment of the present invention, each rᵢⱼₚ on a plate p is normalized by a standard deviation value derived from all the rᵢⱼₚ's on the plate p (step 309), thus resulting in a score for the well (i, j, p) that can be compared across plates p:

scoreᵢⱼₚ=rᵢⱼₚ/(standard deviation value)ₚ.

The standard deviation value may be any appropriate standard deviation value without departing from the spirit and scope of the present invention. For example, the standard deviation value may be a median absolute deviation from median value multiplied by an appropriate constant to produce an unbiased estimate for a Gaussian distribution. Such median absolute deviation from median value and such multiplication constant are known to those in the relevant art and need not be described herein in any detail.

Once the scoreᵢⱼₚ has been developed for each well (i, j, p), all the scoreᵢⱼₚ values for all the wells (i, j, p) may then be compared/organized/ranked and otherwise formatted into an appropriate form, as was described above in connection with step 105 of FIG. 1.

Any particular method may be employed to choose ‘hits’ based on the positionally-corrected scores without departing from the spirit and scope of the present invention. One can, of course, arbitrarily set a cutoff, although it is to be noted that such cutoffs vary and frequently change for a variety of reasons even after they have been set for a given assay. In point of fact, there can be no cutoff that is always successful in distinguishing true hits from false positives. That is, there is always some probability of false positives or false negatives based on any chosen cutoff.

Whatever hit determining device is used in connection with the positionally corrected scores of the present invention, the point is that the better a score is, the more likely it is that the corresponding sample is a true hit. That is, positionally correcting scores in accordance with the present invention provides a better scoring system to increase the probability of finding true hits as one goes down the list from best to worse. Accordingly, it is recommended in connection with the present invention that the best k % of the positionally corrected scores be confirmed. Typically, k is about 1, although k ideally should be greater assuming it can be afforded, remembering that more elaborate assays are both more expensive and time consuming. Importantly, there is no ‘magic cutoff’, only statistically unusual results.

In the foregoing description, it can be seen that the present invention comprises a new and useful statistical algorithm for positionally correcting assay screening data. The algorithm corrects for possible plate positional effects, including transitory positional effects due to quality control problems like clogged tips, reader anomalies, and so forth. The algorithm does not require the use of any blank and/or control values and works in the presence of missing values. The algorithm also standardizes corrected raw values across plates, thus allowing values to be ranked across plates. It should be appreciated that changes could be made to the embodiments described above without departing from the inventive concepts thereof. For example, instead of using a running median smoother, a ‘lowess’ procedure may be employed, as should be appreciated by the relevant public. It should be understood, therefore, that this invention is not limited to the particular embodiments disclosed, but it is intended to cover modifications within the spirit and scope of the present invention as defined by the appended claims.

1-9. (canceled)

10. A method of positionally correcting raw assay data from an assay comprising a plurality of longitudinally oriented plates p, each plate p having a plurality of wells organized into rows i and columns j, each well (i, j, p) having a raw value xijp associated therewith, the raw values xijp comprising the raw assay data, the method comprising deconstructing each raw value xijp of an associated well (i, j, p) into:
a plate effect value representing extraneous effects attributable to the plate p of the well (i, j, p);
a row effect value representing extraneous effects attributable to the row i on the plate p of the well (i, j, p);
a column effect value representing extraneous effects attributable to the column j on the plate p of the well (i, j, p);
a non-additive, interaction effect value representing extraneous positional effects attributable to consistent positional effects beyond the plate, row, and column effects previously determined for the (i, j, p) well on plate p; and
a residual data value that is left over once all the above extraneous effects are taken into account,
the method further comprising employing the residual data value associated with each well (i, j, p) to represent the well (i, j, p) as compared with all other wells (i, j, p) on the plate p.

11. The method of claim 10 wherein the raw assay data is generated from a high throughput screening assay to identify a biologically active agent in a collection of test agents, wherein the assay is subject to positional and systemic effects, the raw assay data is arranged in a three dimensional array, a biologically active agent is identified by identifying a test agent that generates a data point which statistically deviates from other data points in the formatted scored data.

12. The method of claim 10 comprising employing the residual data value associated with each well (i, j, p) to represent the well (i, j, p) as compared with all other wells (i, j, p) on all of the plates p.

13. The method of claim 10 comprising deconstructing each raw value xijp of an associated well (i, j, p) into a plate effect value representing the overall median of all the raw values of the wells on plate p.

14. The method of claim 13 comprising deconstructing each raw value xijp of an associated well (i, j, p) into a row effect value representing the median of all the raw values of the wells for row i on plate p after taking into consideration the plate effect value.

15. The method of claim 13 comprising deconstructing each raw value xijp of an associated well (i, j, p) into a column effect value representing the median of all the raw values of the wells for column j on plate p after taking into consideration the plate effect value.

16. The method of claim 10 comprising deconstructing each raw value xijp of an associated well (i, j, p) into a non-additive, interaction effect value representing an additional possible systematic measurement effect beyond the plate, row, and column effect values previously determined for the (i, j, p) well on plate p.

17. A method of positionally correcting raw assay data from an assay comprising a plurality of longitudinally oriented plates p, each plate p having a plurality of wells organized into rows i and columns j, each well (i, j, p) having a raw value xijp associated therewith, the raw values xijp comprising the raw assay data, the method comprising:
resistantly fitting the raw value xijp for each well (i, j) for each plate p to a row-column additive model:
yijp=μp+R′ip+C′jp+eijp.
where:
yijp=the raw value for the well at row i and column j on plate p;
μp=an overall “average” for plate p;
R′ip=a possible systematic measurement row offset for row i on plate p;
C′jp=a possible systematic measurement column offset for column j on plate p; and
eijp=residual data without taking into account any non-additive interaction offset;
longitudinally (plate-wise) non-linearly smoothing each R′ip and each C′jp;
substituting each un-smoothed R′ip and C′jp value with a corresponding smoothed Rip and Cjp value and adjusting each residual value eijp to an adjusted e′ijp:
yijp=μp+Rip+Cip+e′ijp;
longitudinally (plate-wise) non-linearly smoothing each e′ijp to result in:
yijp=μp+Rip+Cjp+smoothp(e′ijp)+rijp
where each e′ijp is deconstructed into smoothp(e′ijp), a possible systematic non-additive interaction offset for the (i, j) well on plate p, and rijp, residual data left over after taking into account any interaction offset;
wherein each rijp represents a true relative value of the corresponding well (i, j, p) as compared to all other wells (i, j, p) on the plate p.

18. The method of claim 17 wherein the raw assay data is generated from a high throughput screening assay to identify a biologically active agent in a collection of test agents, wherein the assay is subject to positional and systemic effects, the raw assay data is arranged in a three dimensional array, a biologically active agent is identified by identifying a test agent that generates a data point which statistically deviates from other data points in the formatted scored data.

19. The method of claim 17 comprising resistantly fitting the raw value xijp for each plate p to a row-column additive model according to a two way resistant median polish procedure.

20. The method of claim 17 comprising longitudinally (plate-wise) non-linearly smoothing each R′ip and each C′jp according to one of a running median smoother and a lowess procedure.

21. The method of claim 17 comprising longitudinally (plate-wise) non-linearly smoothing each e′ijp according to one of a running median smoother and a lowess procedure.

22. The method of claim 17 further comprising normalizing each rijp to result in a true relative value of the corresponding well (i, j, p) that can be compared to all other wells (i, j, p) on all plates p.

23. The method of claim 22 comprising normalizing each rijp by a standard deviation value derived from all the rijp's on the plate p to result in a score for the well (i, j, p) that can be compared across plates p:
scoreijp=rijp/(standard deviation value)p.

24. The method of claim 23 comprising normalizing each rijp by a median absolute deviation from median value.

25. A computer having computer modules executing thereon for positionally correcting raw assay data from an assay comprising a plurality of longitudinally oriented plates p, each plate p having a plurality of wells organized into rows i and columns j, each well (i, j, p) having a raw value xijp associated therewith, the raw values xijp comprising the raw assay data, the modules comprising a first module deconstructing each raw value xijp of an associated well (i, j, p) into:
a plate effect value representing extraneous effects attributable to the plate p of the well (i, j, p);
a row effect value representing extraneous effects attributable to the row i on the plate p of the well (i, j, p);
a column effect value representing extraneous effects attributable to the column j on the plate p of the well (i, j, p);
a non-additive, interaction effect representing extraneous positional effects attributable to consistent positional effects beyond the plate, row, and column effects determined for the (i, j, p) well on plate p; and
a residual data value that is left over once all the above extraneous effects are taken into account, the computer further comprising a second module employing the residual data value associated with each well (i, j, p) to represent the well (i, j, p) as compared with all other wells (i, j, p) on the plate p.

26. The computer of claim 25 wherein the raw assay data is generated from a high throughput screening assay to identify a biologically active agent in a collection of test agents, wherein a biologically active agent is identified by identifying a test agent that generates residual data value which statistically deviates from other residual data values generated.

27. The computer of claim 25 wherein the second module employs the residual data value associated with each well (i, j, p) to represent the well (i, j, p) as compared with all other wells (i, j, p) on all of the plates p.

28. The computer of claim 25 wherein the first module deconstructs each raw value xijp of an associated well (i, j, p) into a plate effect value representing the overall median of all the raw values of the wells on plate p.

29. The computer of claim 28 wherein the first module deconstructs each raw value xijp of an associated well (i, j, p) into a row effect value representing the median of all the raw values of the wells for row i on plate p after taking into consideration the plate effect value.

30. The computer of claim 28 wherein the first module deconstructs each raw value xijp of an associated well (i, j, p) into a column effect value representing the median of all the raw values of the wells for column j on plate p after taking into consideration the plate effect value.

31. The computer of claim 25 wherein the first module deconstructs each raw value xijp of an associated well (i, j, p) into a non-additive interactive effect value representing possible systematic measurement effect for the (i, j) well on plate p beyond that attributable to the plate, row and column effect values.

32. The computer of claim 25 further comprising an inputting module inputting the raw values xijp into a data structure in a memory of the computer, whereby the first module accesses the raw values xijp from the data structure.

33. A computer having computer modules executing thereon for positionally correcting raw assay data from an assay comprising a plurality of longitudinally oriented plates p, each plate p having a plurality of wells organized into rows i and columns j, each well (i, j, p) having a raw value xijp associated therewith, the raw values xijp comprising the raw assay data, the modules comprising:
a first module resistantly fitting the raw value xijp for each well (i, j) for each plate p to a row-column additive model:
yijp=μp+R′ip+C′jp+eijp.
where:
yijp=the raw value for the well at row i and column j on plate p;
μp=an overall “average” for plate p;
R′ip=a possible systematic measurement row offset for row i on plate p;
C′jp=a possible systematic measurement column offset for column j on plate p; and
eijp=residual data without taking into account any non-additive interaction offset;
a second module longitudinally (plate-wise) non-linearly smoothing each R′ip and each C′jp;
a third module substituting each un-smoothed R′ip and C′jp value with a corresponding smoothed Rip and Cjp value and adjusting each residual value eijp to an adjusted e′ijp:
yijp=μp+Rip+Cip+e′ijp; and
a fourth module longitudinally (plate-wise) non-linearly smoothing each e′ijp to result in:
yijp=μp+Rip+Cjp+smoothp(e′ijp)+rijp
where each e′ijp is deconstructed into smoothp(e′ijp), a possible systematic non-additive interaction offset for the (i, j) well on plate p and rijp residual data value left over after taking into account any non-additive interaction offset;
wherein each rijp represents a true relative value of the corresponding well (i, j, p) as compared to all other wells (i, j, p) on the plate p.

34. The computer of claim 33 wherein the raw assay data is generated from a high throughput screening assay to identify a biologically active agent in a collection of test agents, wherein a biologically active agent is identified by identifying a test agent that generates an rijp which statistically deviates from rijp generated.

35. The computer of claim 33 wherein the first module resistantly fits the raw value xijp for each plate p to a row-column additive model according to a two way resistant median polish procedure.

36. The computer of claim 33 wherein the second module longitudinally (plate-wise) non-linearly smoothes each R′ip and each C′jp according to one of a running median smoother and a lowess procedure.

37. The computer of claim 33 wherein the fourth module longitudinally (plate-wise) non-linearly smoothing each e′ijp according to one of a running median smoother and a lowess procedure.

38. The computer of claim 33 further comprising a fifth module normalizing each rijp to result in a true relative value of the corresponding well (i, j, p) that can be compared to all other wells (i, j, p) on all plates p.

39. The computer of claim 38 wherein the fifth module normalizes each rijp by a standard deviation value derived from all the rijp's on the plate p to result in a score for the well (i, j, p) that can be compared across plates p:
scoreijp=rijp/(standard deviation value)p.

40. The computer of claim 39 wherein the fifth module normalizes each rijp by a median absolute deviation from median value.

41. The computer of claim 33 further comprising an inputting module inputting the raw values xijp into a data structure in a memory of the computer, whereby the first module accesses the raw values xijp from the data structure.

42. A computer-readable medium having computer-executable modules thereon for positionally correcting raw assay data from an assay comprising a plurality of longitudinally oriented plates p, each plate p having a plurality of wells organized into rows i and columns j, each well (i, j, p) having a raw value xijp associated therewith, the raw values xijp comprising the raw assay data, the modules comprising a first module for deconstructing each raw value xijp of an associated well (i, j, p) into:
a plate effect value representing extraneous effects attributable to the plate p of the well (i, j, p);
a row effect value representing extraneous effects attributable to the row i on the plate p of the well (i, j, p);
a column effect value representing extraneous effects attributable to the column j on the plate p of the well (i, j, p);
a non-additive, interaction effect representing extraneous positional effects attributable to systematic positional effects beyond the plate, row, and column effects previously determined for the (i, j, p) well on plate p; and
a residual data value that is left over once all the above extraneous effects are taken into account,
the computer further comprising a second module for employing the residual data value associated with each well (i, j, p) to represent the well (i, j, p) as compared with all other wells (i, j, p) on the plate p.

43. The computer-readable medium of claim 42 wherein the raw assay data is generated from a high throughput screening assay to identify a biologically active agent in a collection of test agents, wherein a biologically active agent is identified by identifying a test agent that generates a residual data value which statistically deviates from the other residual data value generated by the assay.

44. The computer-readable medium of claim 42 wherein the second module employs the residual data value associated with each well (i, j, p) to represent the well (i, j, p) as compared with all other wells (i, j, p) on all of the plates p.

45. The computer-readable medium of claim 42 wherein the first module deconstructs each raw value xijp of an associated well (i, j, p) into a plate effect value representing the overall median of all the raw values of the wells on plate p.

46. The computer-readable medium of claim 45 wherein the first module deconstructs each raw value xijp of an associated well (i, j, p) into a row effect value representing the median of all the raw values of the wells for row i on plate p after taking into consideration the plate effect value.

47. The computer-readable medium of claim 45 wherein the first module deconstructs each raw value xijp of an associated well (i, j, p) into a column effect value representing the median of all the raw values of the wells for column j on plate p after taking into consideration the plate effect value.

48. The computer-readable medium of claim 42 wherein the first module deconstructs each raw value xijp of an associated well (i, j, p) into a non-additive interactive effect value representing a possible systematic measurement effect for the (i, j) well on plate p with respect to (i, j) wells on nearby plates p.

49. The computer-readable medium of claim 42 further comprising an inputting module inputting the raw values xijp into a data structure in a memory of a computer, whereby the first module accesses the raw values xijp from the data structure.

50. A computer-readable medium having computer-executable modules thereon for positionally correcting raw assay data from an assay comprising a plurality of longitudinally oriented plates p, each plate p having a plurality of wells organized into rows i and columns j, each well (i, j, p) having a raw value xijp associated therewith, the raw values xijp comprising the raw assay data, the modules comprising:
a first module for resistantly fitting the raw value xijp for each well (i, j) for each plate p to a row-column additive model:
yijp=μp+R′ip+C′jp+eijp.
where:
yijp=the raw value for the well at row i and column j on plate p;
μp=an overall “average” for plate p;
R′ip=a possible systematic measurement row offset for row i on plate p;
C′jp=a possible systematic measurement column offset for column j on plate p; and
eijp=residual data without taking into account any non-additive interaction offset;
a second module for longitudinally (plate-wise) non-linearly smoothing each R′ip and each C′jp;
a third module for substituting each un-smoothed R′ip and C′jp value with a corresponding smoothed Rip and Cjp value and adjusting each residual value eijp to an adjusted e′ijp:
yijp=μp+Rip+Cip+e′ijp; and
a fourth module for longitudinally (plate-wise) non-linearly smoothing each e′ijp to result in:
yijp=μp+Rip+Cjp+smoothp(e′ijp)+rijp
where each e′ijp is deconstructed into smoothp(e′ijp), a possible systematic non-additive interaction measurement offset for the (i, j) well on plate p, and rijp, residual data left over after taking into account any interaction offset;
wherein each rijp represents a true relative value of the corresponding well (i, j, p) as compared to all other wells (i, j, p) on the plate p.

51. The computer-readable medium of claim 50 wherein the raw assay data is generated from a high throughput screening assay to identify a biologically active agent in a collection of test agents, wherein a biologically active agent is identified by identifying a test agent that generates an rijp which statistically deviates from the rijp generated by the other agents in the assay.

52. The computer-readable medium of claim 50 wherein the first module resistantly fits the raw value xijp for each plate p to a row-column additive model according to a two way resistant median polish procedure.

53. The computer-readable medium of claim 50 wherein the second module longitudinally (plate-wise) non-linearly smoothes each R′ip and each C′jp according to one of a running median smoother and a lowess procedure.

54. The computer-readable medium of claim 50 wherein the fourth module longitudinally (plate-wise) non-linearly smoothing each e′ijp according to one of a running median smoother and a lowess procedure.

55. The computer-readable medium of claim 50 further comprising a fifth module for normalizing each rijp to result in a true relative value of the corresponding well (i, j, p) that can be compared to all other wells (i, j, p) on all plates p.

56. The computer-readable medium of claim 55 wherein the fifth module normalizes each rijp by a standard deviation value derived from all the rijp's on the plate p to result in a score for the well (i, j, p) that can be compared across plates p:
scoreijp=rijp/(standard deviation value)p.

57. The computer-readable medium of claim 56 wherein the fifth module normalizes each rijp by a median absolute deviation from median value.

58. The computer-readable medium of claim 50 further comprising an inputting module for inputting the raw values xijp into a data structure in a memory of a computer, whereby the first module accesses the raw values xijp from the data structure.
